(19) United States
(12) Patent Application Publication
(10) Pub. No.: US 2016/0038468 A1
(43) Pub. Date: Feb. 11, 2016

(54) RIFAXIMIN FOR USE IN THE TREATING OF VAGINAL INFECTIONS

(71) Applicant: ALFA WASSERMANN S.P.A., Alanno (pescara) (IT)

(72) Inventors: Giuseppe Claudio VISCOMI, Bologna (IT); Fiorella CALANNI, Bologna (IT)

(21) Appl. No.: 14/776,731

(22) PCT Filed: Mar. 3, 2014
(86) PCT No.: PCT/IB2014/059400

§ 371 Date: Sep. 14, 2015

Related U.S. Application Data
(60) Provisional application No. 61/794,323, filed on Mar. 15, 2013.

(51) Int. Cl. A61K 31/437; A61K 31/4164; A61K 31/7056

(52) U.S. Cl. CPC A61K 31/437; A61K 31/7056; A61K 31/4164

(57) ABSTRACT

This disclosure relates to rifaximin—for use in the treatment of bacterial vaginal infections. The disclosure also relates to the use of rifaximin for treating infections characterized by the presence of bacteria that may be clindamycin and/or metronidazole resistant. The disclosure also relates to the use of rifaximin to treat patients with vaginal infections who have relapsed following prior treatment or who have bacteria resistant to antibiotics other than rifaximin.

### FIELD OF THE INVENTION

This invention relates to rifaximin for use in the treatment of bacterial vaginal infections. The invention also relates to the use for treating infections characterized by the presence of bacteria that may be clindamycin and/or metronidazole resistant. The invention also relates to the use of rifaximin to treat patients with vaginal infections who have relapsed following prior treatment or who have bacteria resistant to antibiotics other than rifaximin.

### BACKGROUND

Rifaximin (INN, see The Merck Index, XIII ed., 8304, CAS No. 80621-81-4), IUPAC nomenclature 2S, 16Z, 18E, 20S, 21S, 22R, 23R, 24R, 25S, 26S, 27S, 28E)-5,6,21,23,25 pentahydroxy-27-methoxy-2,4,11,16,20,22,24,26-octamethyl-2,7-(epoxypentadeca (1,11,13)trienimine)benzofuro (4,5-e)pyrido(1,2,-a benzimidazole-1,15(2H)dione, 25-acetate) is a semysinthetic antibiotic drug belonging to the rifampicin group, more precisely a pyrido-imidazo-rifamycin described in IT 1154655, whereas EP 0 161 534 describes a production process starting from Rifamycin 0 (The Merck Index XIII ed., 8301).

U.S. Pat. No. 7,045,620, EP 1557421B1, EP 1676847B1, EP 1676848B1, WO2005/044823, WO2006/094662 describe crystalline forms α, β, γ, δ and ε of rifaximin. WO 2008/155728, US 2009/312357 and U.S. Pat. No. 7,709,634 B2 describe processes for obtaining amorphous forms.

WO 2009/108730 describes polymorphous forms of rifaximin named zeta, eta, α-dry, iota, β-1, β-2 and ε-dry.

WO 2011/153444 describes polymorphous forms κ and θ and WO 2011/156897 describes polymorphous forms named APO-1 and APO-2.

Viscomi G. et al in Cryst. Eng Comm., 2008, 10 1074-1081(2008) describes polymorphous α, β, γ, δ, ε, the process for obtaining them and their chemical-physical and biological properties.

Rifaximin is an antibiotic drug active against Gram-positive and Gram-negative bacteria, characterized by a low systemic absorption which is negligible when administered via the oral route. As described by Descombe J. J. et al., Int J Clin Pharmacol Res, 14 (2), 51-56, (1994), rifaximin is known for its antibacterial activity against bacteria, for instance, localized in the gastrointestinal tract causing intestinal infections, diarrhea and irritable bowel syndrome (IBS) and bacterial growth in the small intestine or “small intestinal bacterial overgrowth” (SIBO). Rifaximin is also used to treat patients with Crohn's disease (CD), pancreatic insufficiency, enteritis, and fibromyalgia.

For this characteristic, rifaximin plays a relevant role in the therapy of infectious and inflammatory bowel diseases, both in the acute and in the chronic phase.

The different forms of rifaximin are associated with different levels of systemic absorption. Rifaximin is presently authorized for the treatment of acute and chronic pathologies whose etiology partially or completely is attributable to Gram-positive and Gram-negative intestinal bacteria, such as diarrheic syndromes caused by an altered balance of the intestinal microbial flora such as, for example, summer diarrheas, traveler's diarrhea and enterocolitis. Rifaximin is also useful in the pre- and post-surgical prophylaxis of infectious complications associated with gastroenteric tract surgery; as an adjuvant in hyperammonaemias therapy; and in the reduction of the risk of acute episodes of hepatic encephalopathy.

Rifaximin can also be useful in treating “restless-legs syndrome”; for the prevention of spontaneous bacterial peritonitis in patients affected by hepatic insufficiency; and infections induced by the chronic use of proton pump inhibitors.

Furthermore, the fact that rifaximin is poorly absorbed systemically is advantageous for the aforesaid applications, since rifaximin is not toxic, even at high doses and reduces the incidence of undesired side effects such as, for instance, the selection of antibiotic-resistant bacterial strains and the risk of possible pharmacological interactions.

Rifaximin characteristics make it a compound useful in topical treatments, such as those useful for treating vaginal infections, for example bacterial vaginosis (BV).

Bacterial vaginosis is an extremely frequent pathology, representing 40-50% of all vaginal infections. When it is symptomatic and without complications, bacterial vaginosis is characterized by malodorous vaginal discharges not associated with an inflammatory clinical picture (vaginosis), and is attributed to an alteration of the vaginal ecosystem.

The normal vaginal flora of a healthy woman and the growth inhibition of most pathogenic microorganisms is largely due to the prevailing presence of Lactobacilli, in particular Lactobacillus crispatus and gasseri, which produces hydrogen peroxide and maintains an acid vaginal pH.

In bacterial vaginosis, Lactobacillus bacteria is replaced by an excessive growth, even a thousand times higher than normal values, of facultative anaerobic and aerobic bacteria, mainly represented by Gardnerella vaginalis, which is present in nearly all women affected by bacterial vaginosis; by Mycoplasma hominis; by Gram-negative anaerobic bacteria such as Bacteroides and Prevotella; by anaerobes such as Peptostreptococcus; by Gram-positive anaerobes such as Mobiluncus which is present in 50% of the cases; and by Gram-positive bacilli such as Atopobium vaginale, which is present in 95% of cases of bacterial vaginosis.

Factors predisposing the onset of the disease are mainly present in fertile-aged women. Such predisposition factors include women who regularly use vaginal lavages, smoke and have sexual intercourse with several different partners and are of the black race. On the other hand, taking estroprogestinic drugs seems to play a protective role. Also, there is likely a hormonal component involved in its aetiopathogenesis, since this pathology is mainly found in fertile-aged women.

Bacterial vaginosis can be related to several serious gynecological and obstetrical complications, such as, for instance: pelvic inflammatory disease, a frequent cause of sterility and ectopic pregnancy; infection of surgical injury after gynecologic surgery; premature rupture of the membranes in pregnant women; and premature labor and abortion.

Furthermore, although it is not considered a sexually transmitted disease, bacterial vaginosis is associated with an increased risk of catching sexually transmitted pandemic diseases, including HIV virus infection, both for non-pregnant and pregnant women. Bacterial vaginosis also leads to an increase in the risk of transmission of HIV virus from the mother to the fetus.

The diagnosis of bacterial vaginosis can be based upon clinical and/or microbiological criteria.

The clinical diagnosis is carried out according to Amsel clinical criteria, as described by Amsel R. et al., Am J Med 1983; 74(1): 14-22. The diagnosis is positive when at least three out of the four following symptoms are reported: 1) vaginal discharges which are homogeneous and adhering to the vaginal walls; 2) whiff test positivity (development of “fishy odor” after the addition of 10% potassium hydroxide to vaginal discharge); 3) vaginal pH higher than 4.5; and 4) an amount greater than 20% of clue cells (squamous epithelium vaginal cells coated with bacteria, identified by fresh microscopic examination).

The microbiological diagnosis is based on the calculation of the Nugent score, which includes microscopic examination of vaginal discharges by means of Gram staining. The presence and the quantity of three different vaginal bacterial species is determined. In particular, a low score is obtained if the Lactobacilli concentration is high, the score increases if the presence of Gardnerella and Bacteroidi is ascertained, and the score is even higher if the presence of Mobiluncus is also ascertained. A resulting score between 0 and 3 is representative of vaginal flora of a healthy woman, a score between 4 and 6 indicates that vaginal flora is starting to be altered, and a score between 7 and 10 indicates a certain diagnosis of bacterial vaginosis, as described by Nugent R P et al., J Clin Microbiol 1991, 29(2), 297-301.

Moreover, in recent years further diagnostic molecular techniques have been developed, such as PCR-DGGE and real-time PCR, based upon the sequence analysis of RNA and allowing the identification of a microbial composition of the vaginal ecosystem, as described by Zhou X et al., Microbiology 2004, 150 (Pt8), 2565-2573, and Appl Environ Microbiol 2004, 70(6), 3575-3581. Therefore, these techniques can be directly used to determine the presence of pathogenic agents causing the disease and also to verify the effect of therapy on them from the quantitative point of view.

Although the vaginal infections etiology is not completely understood, treatment has the aim of inducing both a clinical and a microbiological recovery and when possible avoiding relapse infections. Therapy is directed towards reducing pathogenic species and preventing possible disease relapses.

The guidelines of the Center of Disease Control (CDC), 2010, 59, NoRR-12 state that all women affected by bacterial vaginosis, which are symptomatic and non-pregnant, should be treated with antibiotic therapy.

In this regard, the CDC suggests, as a first therapeutic approach, antibiotic treatments such as, for instance: metronidazole, oral tablets 500 mg, twice a day for 7 days; or metronidazole, vaginal gel, 0.75%, an applicator (5 g once a day for 5 days or clindamycin, vaginal cream, 2%, an applicator (5 g) once a day for 7 days.

Both metronidazole and clindamycin, administered either via the systemic route (orally) or via local route (vaginally), are effective at treating bacterial vaginosis. However, the inhibitory action of both active principles against Lactobacillus protective flora, as described by Simoes J A et al., Infect Dis Obstet Gynecol 2001, 9(1), 41-45, limits their efficacy for preventing relapses.

Furthermore, both of the above mentioned antibiotics are associated with systemic side effects, some of them particularly significant, such as, for instance, neurological reactions for metronidazole or pseudomembranose colitis for clindamycin, even when administered via the vaginal route.

Moreover, if repeatedly administered, both metronidazole and clindamycin can induce microbiological resistances not only at the vaginal level, but also at the systemic level, since they are systemically absorbed even after vaginal administration.

EP 0547294 describes compositions containing rifaximin in amounts between 50 and 500 mg which are stated to be useful in treating vaginal infections caused by microorganisms susceptible to rifaximin. In particular, EP 0547294 describes a clinical trial carried out with a preparation of rifaximin vaginal foam and cream, containing 200 mg rifaximin, stating the higher efficacy of foam compared to the cream. This document also describes compositions for treating bacterial vaginosis containing rifaximin in capsules, ovules and tablets and it also describes the antibacterial action of rifaximin against bacteria commonly present in the vaginal discharge. Table 1 of EP 0547294 reports important antibacterial activity of rifaximin against both pathogenic bacteria such as Gardnerella vaginalis, Bacteroides bivious-disiens, Mobiluncus and also against non-pathogenic bacteria such as Lactobacilli.

Activity against Lactobacilli, which, when present, is beneficial for maintaining the healthy vaginal environment, must be considered a detrimental event with regard to therapeutic efficacy. In fact, as already stated, the acid environment generated by Lactobacilli is an essential condition for preventing pathogenic bacteria colonization.

Table 1 of EP 0547292 also shows that rifaximin inhibitory action (MIC₅₀) and MIC₉₀) against Lactobacilli is equal to, or even higher than, its action against pathogenic bacteria, such as, for instance, Gardnerella vaginalis, Mobiluncus spp, Bacteroides bivius-disiens. Thus, when administered via the vaginal route, rifaximin indiscriminately acts on the whole bacterial flora, including Lactobacilli.

Debbia A. et al., J Chemother 20, (2), 186-194, 2008 reports that rifaximin exhibits a time-dependent bacterial activity, and U.S. Ser. No. 13/559,013 describes rifaximin pharmaceutical compositions effective in treating vaginal infections, which maintain adequate levels of Lactobacilli concentration, which is important for the prevention of relapse of vaginal infections. Moreover, U.S. Ser. No. 13/559,013 describes a clinical study wherein rifaximin is efficacious in the treatment of vaginal infections at daily dosage less than 100 mg/day.

In the treatment of vaginal infections it is desirable to have an efficacious treatment in the eradication of essentially all of the vaginal pathogen agents because even a low concentration of pathogen bacteria may lead to a relapse of vaginal infections.

There was a need to have an antimicrobial agent to treat women with vaginal infections, such as bacterial vaginosis, who have relapsed following treatment with an antimicrobrial agent other than rifaximin.

There was a need to have an antibiotic agent efficacious in treating vaginal infections in patients who are resistant to treatment by antibiotics such as clindamycin or metronidazole.

### SUMMARY OF THE INVENTION

The invention relates to rifaximin for use in the treatment of bacterial vaginal infection by administering rifaximin, wherein the bacteria are resistant to an antibiotic other than rifaximin.

The invention also provides an use of rifaximin for treating a bacterial vaginal infection in a patient by administering a pharmaceutically effective amount of rifaximin in combination with one or more additional antibiotics. The bacteria include at least one strain that is resistant to the additional antibiotic(s). In some embodiments, rifaximin is administered in series, sequentially, simultaneously, or in conjunction with the additional antibiotic(s), e.g., clindamycin or metronidazole.

The invention also provides the use of rifaximin for treating a relapse bacterial vaginal infection by administering a pharmaceutically effective amount of rifaximin, wherein the infection was previously treated with one or more antibiotics other than rifaximin. In some embodiments, the bacteria include a strain that is resistant to the antibiotic(s) used to treat the previous infection. In particular embodiments, the previous infection was treated with clindamycin or metronizazole. In some embodiments, the infection is bacterial vaginosis.

The invention also provides the use of rifaximin for preventing a relapse bacterial vaginal infection by administering rifaximin in association with clindamycin or metronidazole in an amount that selectively reduces an amount of vaginal pathogenic bacteria, including Prevotella strains.

In some embodiments of the above identified use, the infection is bacterial vaginosis. In particular embodiments, the resistant bacteria or less susceptible bacteria, include one or more of Prevotella, Anaerococcus, Finegoldia, Peptoniphilus, Anaerococcus, Peptoniphilus, Megasphera, Mobilincus and Atopobium. In additional embodiments. The resistant bacteria include Prevotella, e.g., Prevotella bivia.

In some embodiments, the bacteria are resistant to clindamycin or metronidazole. In some embodiments, the bacteria are less susceptible to clindamycin or metronidazole. In some embodiments, the patient is non responsive to clindamycin or metronidazole.

In further embodiments of the above identified use, a daily dose of rifaximin is administered in an amount from 20 to 2000 mg, preferably less than 500 mg, more preferably, less than 100 mg. In particular embodiments, the dosage form is selected from tablets, coated and uncoated tablets, bioadhesive tablets, controlled release tablet, multi layer tablets, capsules, ointment, cream, gel, foam, and vaginal solutions. In some embodiments, the rifaximin is vaginally administered.

### DETAILED DESCRIPTION

The inventions described herein provide an use of rifaximin for treating vaginal infections, for example, bacterial vaginosis, or relapse vaginal infections, comprising administering to a patient in need of treatment a pharmaceutical composition comprising a therapeutically effective amount of rifaximin. The use according to the invention encompass treating patients who are refractory to other antibiotic treatment and therefore have or are susceptible to relapse bacterial infections. Such prior treatment or treatment with an antibiotic other than rifaximin may include treatment with antibiotics including, but not limited to, clindamycin and metronidazole.

The term “rifaximin” is intended in its broadest sense and includes not only “rifaximin” but also its pharmaceutically acceptable salts, solvates, hydrates, derived enantiomers, polymorphs, amorphous forms, co-crystals and pharmaceutically acceptable complexes, with no limitations.

Rifaximin as present in the pharmaceutical compositions of the invention and may be in any polymorphic form. Preferably, rifaximin is in a poorly soluble form, such as α, β, δ or β stabilized with a polyol, when it is used for treating bacterial vaginosis in order to act locally without systemic absorption. This avoids at the systemic level a potential selection risk of antibiotic-resistant bacterial strains which can occur, even at low plasma concentrations.

By selecting different polymorphs of rifaximin, characterized for having different dissolution and absorption, or a mixture thereof, it is possible to prepare compositions, such as tablets, coated and uncoated tablets, bioadhesive tablets, controlled release tablet, multi layer tablets, capsules, ointment, cream, gel, foam, vaginal solutions with pharmaceutically acceptable excipients prepared according to the technologies well-known in the art.

The solid pharmaceutical compositions of the present invention also include rifaximin microgranules, having rifaximin in an amount less than 500 mg, and one or more of an extragranular excipient including at least one disintegrant. The pharmaceutical composition has selective bactericidal activity against vaginal pathogenic bacteria and maintains or increases the amount of Lactobacilli after a course of treatment. Forms of rifaximin and pharmaceutical compositions thereof are described in U.S. Pat. Nos. 7,045,620; 8,158,781; 8,173,801; 7,902,206; 8,217,054; 7,923,553; 8,158,644; 8,193,196; and 6,140,355 which are all incorporated by reference in their entirety.

The solid pharmaceutical compositions of the present invention are therapeutically effective at treating bacterial infections at rifaximin daily doses from 20 mg to 2000 mg, from 10 mg to 100 mg, from 25 mg to 50 mg, preferably less than 500 mg, more preferably less than 100 mg a day.

The compositions can be administered once or several times a day, without any adverse effect and the composition are well tolerated by the patients.

Rifaximin, metronidazole and clindamycin were compared for testing the antimicrobial susceptibility of bacteria found in the vagina of women with or without bacterial vaginosis using the agar dilution procedure. The method used was the reference method approved by the Clinical and Laboratory Standards Institute (CLSI). The CLSI is considered the gold standard for determining the lowest concentration of an antimicrobial agent that prevents growth of a microorganism in an agar dilution susceptibility test referred to as the minimal inhibitory concentration (MIC).

The following organisms were tested:

Gardnerella vaginalis (107 clinical isolates), Atopobium vaginae (50, clinical isolates), Mobiluncus species (60 clinical isolates, including M. curtisii and mulieris), Prevotella bivia (formerly Bacteroides bivius, n=25), Prevotella timonensis (n=25), Prevotella amnii (n=25), Peptoniphilus harei, Peptoniphilus lacrimalis, Anaerococcus tetradius, Finegoldia magna, and Megasphaera-like bacteria (for a total of 100 equally divided).

60 Isolates of Mobiluncus species were planned to be tested for susceptibility, however only 40 unique isolates recovered over the past 3 years from vaginal samples were available for inclusion in this study. In order to provide a more in depth evaluation of the susceptibility of microorganisms associated with bacterial vaginosis, 25 isolates of Megasphaera-like and 62 isolates of Atopobium vaginae were included in this evaluation. Both of these microorganisms have been found to be highly related to bacterial vaginosis, and Megasphaera-like microbes have been linked with both preterm delivery and recurrence of bacterial vaginosis following treatment.

Bacteroides fragilis was not included in this evaluation since detection of this organism in isolates from the vagina is very rare. Among a group of 207 women for whom detailed culture work was performed, anaerobic Gram negative rods were recovered from all women. The most common Bacteroides was B. ureolyticus and a small handful of other species including B. ovatus, B. splanchnicus and one B. uniformis were detected. Therefore, using contemporary methods for careful identification of anaerobic Gram negative rods, B. fragilis cannot be considered a member of the vaginal flora even among women with bacterial vaginosis.

The minimal inhibitory concentration (MIC) values obtained for the ATCC strains of B. fragilis, B. thetaiotaomicron, and Clostridium difficile met the criteria set in the CLSI manual for each antimicrobial agent. If the MIC values of the control strains did not fall within the ranges the test organisms were repeated.

A total of 411 unique microbial isolates recovered from the human vagina of US women from a period of time from 2009 to 2012 were tested for antimicrobial susceptibility by the agar dilution method. A total of 13 analytical runs were conducted in order to analyze all of the samples. See, Example 1.

The 411 vaginal bacterial vaginosis (BV) related organisms were tested for minimal inhibitory concentration (MIC) against three antibiotics (clindamycin, metronidazole and rifaximin). The MIC ranges are shown in Table 1.

Table 1 demonstrates the higher susceptibility of pathogenic strains such as Prevotella, Anaerococcus, Finegoldia, Peptoniphilus and Atopobium strains, to rifaximin in respect to clindamycin.

Moreover Table 1, demonstrates the higher susceptibility of pathogenic strains such as Prevotella, Anaerococcus, Peptoniphilus, Atopobium, Megasphera and Mobiluncus strains, to rifaximin, in respect to metronidazole.

Table 2 contains the susceptibility and resistance of the organisms.

The MIC distribution of rifaximin, clindamycin and metronidazole are presented in Table 3a, Table 3b and Table 3c.

TABLE 1
Ranges of Minimal Inhibitory Concentration (MICS) for bacterial
vaginosis (BV) related organisms
MIC (μg/ml)

| | # | Anti- | | | |
|---|---|---|---|---|---|
| | tes- | microbial | | | |
| Species | ted | Agent | Range | 50% | 90% |
| Gardnerella | 107 | Clindamycin | 0.015-0.5 | 0.125 | 0.25 |
| vaginalis | | Metronidazole | 2 −> 128 | 64 | >128 |
| | | Rifaximin | 0.125 −> 128 | 2 | >128 |
| Prevotella | 33 | Clindamycin | 0.03-128 | 0.03 | 0.03 |
| amnii | | Metronidazole | 0.25-2 | 1 | 2 |
| | | Rifaximin | 0.015-0.03 | 0.015 | 0.03 |
| Prevotella | 34 | Clindamycin | 0.03 −> 128 | >128 | >128 |
| bivia | | Metronidazole | 4-16 | 8 | 16 |
| | | Rifaximin | 0.125-1 | 0.25 | 0.5 |
| Prevotella | 33 | Clindamycin | 0.03 −> 128 | 0.03 | >128 |
| timonensis | | Metronidazole | 1 −>128 | 2 | 4 |
| | | Rifaximin | 0.00375-0.06 | 0.015 | 0.015 |
| Anaero- | 21 | Clindamycin | 0.03 −> 128 | 2 | >128 |
| coccus | | Metronidazole | 0.5-128 | 1 | 1 |
| tetradius | | Rifaximin | 0.00375-0.125 | 0.06 | 0.06 |
| Finegoldia | 20 | Clindamycin | 0.03 −> 128 | 0.5 | 128 |
| magna | | Metronidazole | 0.5-4 | 2 | 4 |
| | | Rifaximin | 0.125-16 | 4 | 16 |
| Peptoni- | 23 | Clindamycin | 0.125 −> 128 | 0.5 | 1 |
| philus | | Metronidazole | 1-4 | 1 | 2 |
| harei | | Rifaximin | 0.00375-0.03 | 0.0075 | 0.015 |
| Peptoni- | 20 | Clindamycin | 0.06 −> 128 | 0.25 | >128 |
| philus | | Metronidazole | 0.5-4 | 1 | 4 |
| lacrimalis | | Rifaximin | 0.00375-0.03 | 0.015 | 0.015 |
| Atopobium | 62 | Clindamycin | 0.03 −> 128 | 0.06 | 0.25 |
| vaginae | | Metronidazole | 4 −> 128 | 128 | >128 |
| | | Rifaximin | 0.00375-0.25 | 0.00375 | 0.015 |
| Megas- | 25 | Clindamycin | 0.03-0.125 | 0.03 | 0.03 |
| phaera- | | Metronidazole | 0.125-0.25 | 0.25 | 0.25 |
| like | | Rifaximin | 0.0075-0.015 | 0.015 | 0.015 |
| bacteria | | | | | |
| Mobiluncus | 40 | Clindamycin | 0.03 −> 128 | 0.125 | 0.25 |
| all species^{a} | | Metronidazole | 2 −> 128 | 8 | >128 |
| | | Rifaximin | 0.0075-0.03 | 0.0075 | 0.015 |

^{a}includes 14 M. curtisii, 25 M. mulieris and 1 Mobiluncus spp.

TABLE 2
Susceptibility and Resistance for bacterial vaginosis (BV) related
organisms
MIC (μg/ml)

| | # | Antimicr. | | Inter- | |
|---|---|---|---|---|---|
| Species | tested | Agent | Susceptible | mediate | Resistant |
| Gardnerella | 100 | Clindamycin | 100 (100) | 0 | 0 |
| vaginalis | | Metronidazole | 15 (15) | 16 (16) | 69 (69) |
| | | Rifaximin | 89 (89) | | 11 (11)^{a} |
| Prevotella amnii | 33 | Clindamycin | 32 (97) | 0 | 1 (3) |
| | | Metronidazole | 33 (100) | 0 | 0 |
| | | Rifaximin | 33 (100) | | 0 ^{b} |
| Prevotella bivia | 34 | Clindamycin | 9 (26) | 0 | 25 (74) |
| | | Metronidazole | 21 (62) | 13 (38) | 0 |
| | | Rifaximin | 34 (100) | 0 | 0 ^{a} |
| Prevotella | 33 | Clindamycin | 19 (58) | 0 | 14 (42) |
| timonensis | | Metronidazole | 32 (97) | 0 | 1 (3) |
| | | Rifaximin | 33 (100) | | 0 ^{c} |
| Anaerococcus | 21 | Clindamycin | 16 (76) | 1 (5) | 4 (19) |
| tetradius | | Metronidazole | 20 (95) | 0 | 1 (5) |
| | | Rifaximin | 21 (100) | | 0 ^{c} |
| Finegoldia | 20 | Clindamycin | 12 (60) | 2 (10) | 6 (30) |
| magna | | Metronidazole | 20 (100) | 0 | 0 |
| | | Rifaximin | 20 (100) | | 0 ^{a} |
| Peptoniphilus | 23 | Clindamycin | 21 (91) | 0 | 2 (9) |
| harei | | Metronidazole | 23 (100) | 0 | 0 |
| | | Rifaximin | 23 (100) | | 0 ^{c} |
| Peptoniphilus | 20 | Clindamycin | 14 (70) | 0 | 6 (30) |
| lacrimalis | | Metronidazole | 20 (100) | 0 | 0 |
| | | Rifaximin | 20 (100) | | 0 ^{c} |
| Atopobium | 62 | Clindamycin | 59 (95) | 0 | 3 (5) |
| vaginae | | Metronidazole | 3 (5) | 5 (8) | 54 (87) |
| | | Rifaximin | 62 (100) | | 0 ^{d} |
| Megasphaera- | 25 | Clindamycin | 25 (100) | 0 | 0 |
| like bacteria | | Metronidazole | 25 (100) | 0 | 0 |
| | | Rifaximin | 25 (100) | | 0 ^{d} |
| Mobiluncus all | 40 | Clindamycin | 38 (95) | 0 | 2 (5) |
| species^{e} | | Metronidazole | 22 (55) | 1 (3) | 17 (42) |
| | | Rifaximin | 40 (100) | | 0 |

TABLE 3a
Minimal Inhibitory Concentration (MIC) distribution of Rifaximin
for bacterial vaginosis (BV) related organisms

| Bacterial | | Number of strains with indicated MIC (μg/ml) | | | | | | | | | | | | | |
|---|---|---|---|---|---|---|---|---|---|---|---|---|---|---|---|
| species | Samples | 0.00375 | 0.0075 | 0.015 | 0.03 | 0.06 | 0.125 | 0.25 | 0.5 | 1 | 2 | 4 | 8 | 16 | >128 |
| G. vaginalis | 100 | | | | | | 2 | 6 | 12 | 28 | 29 | 9 | 3 | | 11 |
| A. tetradius | 21 | 1 | | 3 | 1 | 15 | 1 | | | | | | | | |
| F. magna | 20 | | | | | | 1 | 1 | | | 2 | 9 | 4 | 3 | |
| P. harei | 23 | 1 | 15 | 6 | 1 | | | | | | | | | | |
| P. lacrimalis | 20 | 2 | 5 | 12 | 1 | | | | | | | | | | |
| A. vaginae | 62 | 43 | 10 | 5 | | 1 | 2 | 1 | | | | | | | |
| M. -like | 25 | | 3 | 22 | | | | | | | | | | | |
| bacteria | | | | | | | | | | | | | | | |
| M. curtisii | 14 | | | 12 | 2 | | | | | | | | | | |
| M. mulieris | 25 | | 22 | 3 | | | | | | | | | | | |
| P. amnii | 33 | | | 19 | 14 | | | | | | | | | | |
| P. bivia | 34 | | | | | | 10 | 20 | 3 | 1 | | | | | |
| P. Timonensis | 33 | 1 | 2 | 27 | 2 | 1 | | | | | | | | | |

TABLE 3b
Minimal Inhibitory Concentration (MIC) distribution of Clindamycin
for bacterial vaginosis (BV) related organisms

| Bacterial | | Number of strains with indicated MIC (μg/ml) | | | | | | | | | | | | | | |
|---|---|---|---|---|---|---|---|---|---|---|---|---|---|---|---|---|
| species | Samples | 0.015 | 0.03 | 0.06 | 0.125 | 0.25 | 0.5 | 1 | 2 | 4 | 8 | 16 | 32 | 64 | 128 | >128 |
| G. vaginalis | 100 | 1 | 11 | 25 | 41 | 21 | 1 | | | | | | | | | |
| A. tetradius | 21 | | 1 | | | 1 | | 2 | 12 | 1 | 1 | | | | | 3 |
| F. magna | 20 | | 1 | | | 6 | 4 | | | 3 | 2 | | | | 2 | 2 |
| P. harei | 23 | | | | 5 | 7 | 4 | 5 | | | | | | | | 2 |
| P. lacrimalis | 20 | | | 1 | 4 | 9 | | | | | 1 | | | | 2 | 3 |
| A. vaginae | 62 | | 28 | 10 | 16 | 4 | | 1 | | | 1 | 1 | | | | 1 |
| M. -like | 25 | | 24 | | 1 | | | | | | | | | | | |
| bacteria | | | | | | | | | | | | | | | | |
| M. curtisii | 14 | | | 3 | 4 | 5 | | | | | | | 1 | | | 1 |
| M. mulieris | 25 | | 1 | 12 | 12 | | | | | | | | | | | |
| P. amnii | 33 | | 32 | | | | | | | | | | | | 1 | |
| P. bivia | 34 | | 8 | 1 | | | | | | | | | | | | 25 |
| P. timonensis | 33 | | 17 | 2 | | | | | | | | | | | 1 | 13 |

TABLE 3c
Minimal Inhibitory Concentration (MIC) distribution of Metronidazole
for bacterial vaginosis (BV) related organisms

| Bacterial | | Number of strains with indicated MIC (μg/ml) | | | | | | | | | | | | | |
|---|---|---|---|---|---|---|---|---|---|---|---|---|---|---|---|
| species | Samples | 0.03 | 0.06 | 0.125 | 0.25 | 0.5 | 1 | 2 | 4 | 8 | 16 | 32 | 64 | 128 | >128 |
| G. vaginalis | 100 | | | | | | | 1 | 2 | 12 | 16 | 14 | 15 | 6 | 34 |
| A. tetradius | 21 | | | | | 2 | 18 | | | | | | | 1 | |
| F. magna | 20 | | | | | 2 | 3 | 6 | 9 | | | | | | |
| P. harei | 23 | | | | | | 13 | 9 | 1 | | | | | | |
| P. lacrimalis | 20 | | | | | 1 | 12 | 4 | 3 | | | | | | |
| A. vaginae | 62 | | | | | | | | 1 | 2 | 5 | 10 | 8 | 12 | 24 |
| M. bacteria | 25 | | | 5 | 20 | | | | | | | | | | |
| M. curtisii | 14 | | | | | | | | | | | | 3 | | 11 |
| M. mulieris | 25 | | | | | | | 5 | 7 | 9 | 1 | | 1 | | 2 |
| P. amnii | 33 | | | | 1 | 7 | 21 | 4 | | | | | | | |
| P. bivia | 34 | | | | | | | | 15 | 6 | 13 | | | | |
| P. timonensis | 33 | | | | | | 2 | 22 | 8 | | | | | | 1 |

The taxonomic status of many of the microorganisms associated with bacterial vaginosis has changed in the past several years. The data generated for this group of bacteria suggest that clindamycin resistance is increasing among obligately anaerobic bacteria, and that most vaginal isolates of Prevotella bivia are now resistant to this antimicrobial agent. Metronidazole, which remain the most commonly used antimicrobic agent for the treatment of bacterial vaginosis has limited activity against either G vaginalis or Atopobium vaginae, both of which are uniformly present among women with bacterial vaginosis. By comparison, rifaximin had activity against nearly 90% of G vaginalis strains and 62 strains of Atopobium vaginae. Although fewer isolates of Mobiluncus species were available for inclusion in this study than had been planned, the data generated suggested that both clindamycin and rifaximin had activity on this organism, whereas metronidazole was substantially less active against this organism.

Megasphaera-like bacteria have been described as being strongly associated with vaginal infections bacterial vaginosis using culture independent methods and were until recently thought to be noncultivable.

The MICs values for all pathogens analyzed in the experimental study of the present invention demonstrate that rifaximin is more efficacious in the treatment of all pathogen vaginal bacteria, in particular when also Prevotella strains are present, in comparison to metronidazole and clindamycin

In one embodiment, the use of rifaximin for treating bacterial vaginosis comprises administering to a patient in need thereof, a therapeutically effective amount of rifaxim in wherein Prevotella, Anaerococcus, Finegoldia, Peptoniphilus, Anaerococcus, Peptoniphilus, Megasphera, Mobilincus and Atopobium strains, are present.

In one particular embodiment the infection is characterized by the presence of Prevotella bivia, formerly Bacteroids bivius. Another use of rifaximin for treating bacterial vaginosis involves a combination therapy such that a relapse vaginal infection, such as for example a bacterial vaginosis infection, that was initially treated by clindamycin is subsequently treated with rifaximin. A further use of rifaximin involves a combination therapy such that a relapse vaginal infection, for example, a bacterial vaginosis infection, wherein metronidazole is initially administered and rifaximin is subsequently administered.

Rifaximin may be administered following prior antibiotics therapy or in conjunction with other antibiotic therapy. Rifaximin and other antibiotics may be administered separately or together and administration may be serially, separately, or simultaneously. Rifaximin and another antibiotics may be administered so that they are both active at the same time, or in other embodiments so that they are active at different times. As used herein “association with” means that a patient is treated with both rifaximin and another antibiotic so that both rifaximin and the other antibiotics are active concurrently.

In another embodiment, the use of rifaximin for treating vaginal infections comprises administering rifaximin at daily dosage from 20 mg to 2000 mg, from 25 mg to 200 mg, from 50 mg to 75 mg, preferably less than 100 mg, in form of tablets, coated and uncoated tablets, bioadhesive tablets, controlled release tablet, multi layer tablets, capsules, ointment, cream, gel, foam, vaginal solutions for the treatment of vaginal infections.

In some embodiments of the use of rifaximin when at least one or more of the bacteria strains Prevotella, Anaerococcus, Finegoldia, Peptoniphilus, Anaerococcus, Peptoniphilus, Megasphera, Mobilincus and Atopobium, are present in vaginal specimens from the patient.

In another embodiment, the use of rifaximin comprises administering rifaximin at daily dosage from 20 mg to 2000 mg, from 25 mg to 200 mg, from 50 mg to 75 mg, preferably less than 100 mg in association with or after clindamycin treatment wherein the infection is characterized by the presence of Prevotella strains, in particular Prevotella bivia (formerly Bacteriods bivius) are present.

In another embodiment, the use of rifaximin comprises the administration of rifaximin for treating a relapse bacterial vaginal infection in a patient after said patient had been treated with clindamycin.

Another embodiment is the use of rifaximin for treating patients with bacterial vaginal infection in need thereof comprising administering to the patient rifaximin in association with clindamycin.

Another embodiment of the invention is the use of rifaximin for treating patients with a relapse bacterial vaginal infection in need thereof comprising administering to the patient rifaximin after metronidazole treatment. In a specific embodiment is the use of rifaximin for treating a patient with a relapse bacterial vaginal infection, the previous infection was treated with metronidazole.

Another embodiment is the use of rifaximin in the bacterial vaginosis in association with metronidazole.

Example 1 describes the use of rifaximin adopted to evaluate the in vitro antimicrobial susceptibility of rifaximin, metronidazole and clindamycin against about 400 clinical isolates recovered by vaginal culture according to the guidelines of the Clinical and Laboratory Standards Institute (CLSI) reference agar dilution method.

The data obtained are reported in the Tables 4-14 wherein the MIC values are reported for rifaximin, metronidazole, and clindamycin for each isolates.

### Example 1

A total of 411 unique microbial isolates recovered from the human vagina from 2009-2012 were tested for antimicrobial susceptibility by the agar dilution method. This procedure followed the guidelines of the Clinical Laboratory Standard Institute reference agar dilution method (CLSI).

A total of 13 analytical runs were conducted in order to analyze all of the samples. The following organisms were tested:

Gardnerella vaginalis (107 clinical isolates), Atopobium vaginae (50, clinical isolates), Mobiluncus species (60 clinical isolates, including M. curtisii and mulieris), Prevotella bivia (formerly Bacteroides bivius, n=25), Prevotella timonensis (n=25), Prevotella amnii (n=25), Peptoniphilus harei, Peptoniphilus lacrimalis, Anaerococcus tetradius, Finegoldia magna, and Megasphaera-like bacteria (for a total of 100 equally divided).

### A) Weighing Antimicrobial Powders

Each lot of drug may differ in the amount of activity, therefore standardized antimicrobial solutions were used based on the potency for each lot of drug. The manufacturer provided purity of the drug measured by high performance liquid chromatography (HPLC), water content measured by Karl Fisher analysis or by weight loss on drying, and the salt/counter-ion fraction if the compound is supplied as a salt instead of free acid or base. The potency may be expressed as a percentage or in units of μg/mg (w/w).

To calculate the potency using the manufacturer's certificate of analysis data, the following formula was used:

Potency=(Assay purity)×(Active fraction)×(1-water content)

Either of the following formulas below maybe used to determine the amount of powder or diluent needed for a standard solution:

Weight(mg)=Volume(ml)×Concentration(μg/ml)
Potency (μg/mg)
Or

Volume(ml)=Weight(mg)×Potency(μg/mg)
Concentration (μg/ml)

### B) Preparing the Stock Solution of Drug

The stock solution has been prepared at concentrations of at least 1000 μg/ml or ten times the highest concentration to be tested, whichever is greater.

Example: 1280 μg/ml.

Some drugs must be dissolved in solvents other than water. In such cases a minimum amount of solvent should be used to solubilize the antimicrobial powder and once in solution the final stock concentration can be made with water.

Metronidazole: Dimethyl sulfoxide was used as the solvent and water as the diluent

Clindamycin: water was used for solvent and diluent

Rifaximin: methanol was used as the solvent and 0.45% SDS as the diluents.

### C) Storage of Stock

Small volumes of the stock solutions were dispensed into sterile glass, polypropylene, polystyrene, or polyethylene vials, with tight seals and stored at <−60° C. The drugs are stable for at least 6 months without significant loss of activity. Enough stock drug was aliquoted to be used for set of organisms (32 isolates including three controls). Stock solutions were not refrozen.

The concentrations of drug to be tested was from 0.00375 to 128 μg/ml.

### D) Making Dilution of Drug the Day Before Testing

16 sterile tubes were labeled with the intermediate concentration (0.0375 to 1280 μg/ml).

According to the table below, the drug was diluted with sterile deionized water as the diluent.

| | | | Intermediate | Final concentration |
|---|---|---|---|---|
| Concentration | Drug | Diluent | conc. | in agar plates |
| of drug | volume | volume | (μg/ml) | (μg/ml) |
| 2000 μg/ml | 6.4 ml | 3.6 ml | 1280 | 128 |
| 1280 | 2 | 2 | 640 | 64 |
| 1280 | 1 | 3 | 320 | 32 |
| 1280 | 1 | 7 | 160 | 16 |
| 160 | 2 | 2 | 80 | 8 |
| 160 | 1 | 3 | 40 | 4 |
| 160 | 1 | 7 | 20 | 2 |
| 20 | 2 | 2 | 10 | 1 |
| 20 | 1 | 3 | 5 | 0.5 |
| 20 | 1 | 7 | 2.5 | 0.25 |
| 2.5 | 2 | 2 | 1.25 | 0.125 |
| 2.5 | 1 | 3 | 0.6 | 0.06 |
| 2.5 | 1 | 7 | 0.3 | 0.03 |
| 0.3 | 2 | 2 | 0.15 | 0.015 |
| 0.3 | 1 | 3 | 0.075 | 0.0075 |
| 0.3 | 1 | 7 | 0.0375 | 0.00375 |

A second set of 30 ml sterile tubes was labeled with the final concentrations (0.00375 to 128 μg/ml).

2.0 MI from each of the tubes was transferred with the intermediate concentration into the second set of tubes with the final concentration. (Brucella agar was then added to these tubes. See next section).

### E) Pouring Agar Dilution Plates the Day Before Testing

One Petri dish was labeled for each concentration of drug (0.00375 to 128 μg/ml). Also two Petri dishes were labeled with “start” and “end” for the control plates that do not contain any drug. There was a total of 18 Petri dishes for each drug.

Brucella agar was prepared and supplemented with hem in, and Vitamin K1. The agar was autoclaved and placed in a 50° C. water bath until the temperature of the agar equilibrated to 50° C. (1080 ml of Brucella agar was used for 3 drugs with 16 dilutions plus 2 control plates per drug). The laked sheep blood was added to a final concentration of 5% and mixed to incorporate the sheep blood into the agar. A sterile pipette was used to transfer 18 ml of agar to the tubes containing 2 ml of the drug solution. The solution was gently mixed and poured into the corresponding Petri dish. the liquid agar in the Petri dish was immediately flamed to eliminate any bubbles that formed.

The plates were stored at 4° C. after the agar solidified.

After one day, the day of testing, the plates were dried by placing them with the lids ajar in an incubator for approximately 30 minutes.

### F) Inoculum Preparation for Testing

Each isolate to be tested was previously isolated from vaginal specimens and identified to the species level and stocked in litmus milk and stored at −80° C.

Control organisms:

ATCC 25285
Bacteroides fragilis
ATCC 29741
Bacteroides thetaiotaomicron
ATCC 700057
Clostridium difficile

The isolates were removed from the freezer and inoculated onto Brucella agar supplemented with 5% sheep blood and incubated for 2-4 days at 37° C., in an anaerobic atmosphere. The isolates were subcultured 2 days prior to inoculation onto the drug infused agar.

On the day of testing the organisms a suspension was made in Brucella broth to a turbidity equal to 0.5 McFarland standard.

### G) Inoculation of Agar Dilution Plates

A Steer's replicator was used to deposit approximately 1 to 2 μL onto the agar surface giving a final concentration of 105 CFU per spot. A map of the replicator was prepared and the isolate numbers were recorded for the corresponding wells. Approximately 300 μL of each organism suspension was transferred into the wells of a sterile replicator. The inoculum was applied onto the surface of each plate starting with the plate labeled “start”, followed by the lowest to the highest concentration and ending with the plate labeled “end”. The plates were incubated in an anaerobic chamber or anaerobic jar at 37° C. for 48 hours.

### Reading Agar Dilution Plates

The control plates were read first to confirm growth and to look for possible contamination.

Cross contamination was checked between wells.

The MIC endpoint was read on the test plate. The MIC is that concentration at which a marked reduction occurs in the appearance of growth on the test plate as compared to the amount that of growth on the control plate. Examples of a marked change in growth include a change from confluent growth to a haze, less than 10 tiny colonies, or one to three normal sized colonies. The illustrated figures found in the CLSI manual should be used as a guide.

### Quality Control

The MIC values obtained for the ATCC strains of B. fragilis, B. thetaiotaomicron, and Clostridium difficile when tested in parallel with the test organisms must fall within the acceptable range reported in Clinical and Laboratory Standard Institute manual (CLSI manual) for each antimicrobial agent. If the MIC values of the control strains do not fall within the ranges the test organisms must be repeated. Acceptable Ranges of MIC (μg/ml) for Control Strains for Agar Dilution Testing from CLSI, Table 5 are:

| | B. fragilis, | B. thetaiotaomicron, | Clostridium difficile, |
|---|---|---|---|
| Drug | ATCC 25285 | ATCC 29741 | ATCC 700057 |
| Clindamycin | 0.5-2 | 2-8 | 2-8 |
| Metronidazole | 0.25-1 | 0.5-2 | 0.125-0.5 |
| Rifaximin | No ranges | No ranges | 0.0039-0.0156 |

Endpoint interpretation was monitored periodically to minimize variation in the interpretation of MIC endpoints among observers.

### Reporting of MIC

A bacterial strain was defined as resistant to rifaximin if the MIC is >32 μg/ml or 8×MIC for the most sensitive pathogens.

Resistance to metronidazole is >32 μg/ml

Resistance to clindamycin is >8 μg/ml

TABLE 4
Minimal Inhibitory Concentration (MIC) for 100 isolates of Gardnerella
vaginalis recovered from the vagina between 2009-2012

| Isolate | | Rifaximin | Clindamycin | Metronidazole |
|---|---|---|---|---|
| 1-001 V3 | 8 | 1 | 0.06 | 32 |
| 1-004 V1 | 3 | 0.5 | 0.125 | 16 |
| 1-006 V1 | 11 | 2 | 0.06 | >128 |
| 1-008 V1 | 6A | 2 | 0.125 | >128 |
| 1-008 V1 | 6B | 1 | 0.125 | 128 |
| 1-010 V1 | 6 | >128 | 0.125 | >128 |
| 1-021 V1 | 1 | 0.25 | 0.06 | >128 |
| 1-067 v1 | 3 | 4 | 0.125 | 128 |
| 1-067 V3 | a | 4 | 0.125 | >128 |
| 1-070 V3 | 5 | 2 | 0.25 | 8 |
| 1-071 v1 | 1 | 2 | 0.25 | 32 |
| 1-071 V3 | 6 | >128 | 0.03 | >128 |
| 1-073 V1 | 1 | 4 | 0.125 | >128 |
| 1-073 V1 | 2 | >128 | 0.125 | 32 |
| 1-073 V3 | 4 | 1 | 0.06 | 32 |
| 1-074 V1 | 1 | 2 | 0.125 | 64 |
| 1-075 V1 | 3 | 8 | 0.25 | 64 |
| 1-076 V1 | 4 | >128 | 0.125 | 8 |
| 1-076 V3 | 1 | 2 | 0.125 | 64 |
| 1-077 V3 | a | 0.25 | 0.125 | 32 |
| 1-078 V3 | 1 | 2 | 0.25 | 128 |
| 1-080 V1 | a | 2 | 0.06 | 16 |
| 1-080 V1 | e | 2 | 0.06 | >128 |
| 1-081 V1 | a | 2 | 0.125 | 64 |
| 1-081 V1 | b | 1 | 0.25 | 32 |
| 1-081 V1 | c | 2 | 0.125 | >128 |
| 1-082 V1 | 2 | 4 | 0.25 | 8 |
| 1-082 V3 | 1 | 1 | 0.06 | 32 |
| 1-083 V1 | a | 2 | 0.25 | 4 |
| 1-084 V1 | 1 | 0.25 | 0.03 | 16 |
| 1-084 V3 | 2 | 0.25 | 0.125 | >128 |
| 1-085 V1 | 1 | 2 | 0.25 | >128 |
| 1-085 V3 | 1 | 1 | 0.25 | >128 |
| 1-086 V1 | 3 | 4 | 0.125 | 64 |
| 1-086 V3 | 4 | 4 | 0.06 | 32 |
| 1-087 V1 | 1 | 1 | 0.06 | 4 |
| 1-087 V3 | 2 | 0.25 | 0.125 | 32 |
| 1-090 V1 | 1 | 4 | 0.03 | >128 |
| 1-090 V1 | 1A | 1 | 0.03 | 32 |
| 1-090 V3 | 1 | 2 | 0.03 | >128 |
| 1-092 V1 | a | 1 | 0.125 | 128 |
| 1-094 V1 | 5 | 2 | 0.25 | 64 |
| 1-094 V3 | 1 | 8 | 0.06 | >128 |
| 1-095 V1 | 1 | 1 | 0.125 | 32 |
| 1-097 V1 | 2 | 2 | 0.5 | >128 |
| 1-097 V3 | a | 2 | 0.03 | 16 |
| 1-098 V1 | a | 4 | 0.125 | 16 |
| 1-100 V1 | 3-A | 2 | 0.25 | 128 |
| 1-100 V3 | 1 | 2 | 0.25 | >128 |
| 1-101 V1 | 1 | 2 | 0.06 | 32 |
| 1-102 V1 | 4 | 0.5 | 0.125 | >128 |
| 1-102 V3 | a | 1 | 0.06 | 32 |
| 1-102 V3 | b | 0.5 | 0.25 | 64 |
| 1-102 V3 | m | 1 | 0.06 | 128 |
| 1-103 V1 | 1A | 1 | 0.125 | 16 |
| 1-103 V1 | 1B | 0.125 | 0.125 | 16 |
| 1-103 V3 | 1 | 1 | 0.125 | 64 |
| 1-106 V1 | 1 | 0.5 | 0.125 | 8 |
| 1-107 V1 | 3 | 1 | 0.25 | 8 |
| 1-109 V1 | 1 | 1 | 0.06 | 64 |
| 1-109 V1 | 2 | 1 | 0.125 | 16 |
| 1-110 V2 | 3A | 1 | 0.03 | >128 |
| 1-117 V1 | 1 | 4 | 0.06 | 64 |
| 1-118 V3 | 3A | 2 | 0.125 | >128 |
| 1-119 V3 | 4 | >128 | 0.06 | >128 |
| 1-120 V1 | 1 | 2 | 0.125 | 64 |
| 1-120 V3 | 6 | 2 | 0.06 | 16 |
| 1-121 V1 | 1 | 1 | 0.125 | 16 |
| 1-121 V2 | 4A | >128 | 0.015 | >128 |
| 1-121 V2 | 4B | >128 | 0.03 | 8 |
| 1-123 V2 | 1 | 2 | 0.06 | 64 |
| 1-125 V1 | 1 | 2 | 0.25 | 64 |
| 1-125 V3 | 1 | >128 | 0.06 | 16 |
| 1-126 V1 | 2 | 2 | 0.06 | >128 |
| 1-126 V1 | 1A | 1 | 0.03 | >128 |
| 1-126 V2 | 6 | 1 | 0.06 | >128 |
| 1-127 V1 | 5 | 1 | 0.125 | 32 |
| 1-128 V1 | 1 | >128 | 0.03 | 64 |
| 1-128 V1 | 2 | 1 | 0.06 | 16 |
| 1-129 V2 | 4 | 0.5 | 0.25 | 8 |
| 1-130 V1 | 1 | 1 | 0.125 | 16 |
| 1-130 V3 | 1a | 0.5 | 0.125 | 2 |
| 1-131 V1 | 1 | 1 | 0.125 | >128 |
| 1-131 V3 | 2 | 0.5 | 0.125 | 8 |
| 1-134 V1 | a | 2 | 0.25 | >128 |
| 1-134 V1 | b | 2 | 0.125 | >128 |
| 1-134 V3 | 4 | 0.5 | 0.125 | >128 |
| 1-136 V1 | 1 | 1 | 0.25 | 8 |
| 1-136 V3 | 6 | 0.5 | 0.06 | >128 |
| 1-137 V1 | 2 | 2 | 0.25 | >128 |
| 1-139 V1 | 1 | 0.5 | 0.125 | 8 |
| 1-139 V1 | 2A | >128 | 0.125 | >128 |
| 1-140 v1 | 3 | 0.5 | 0.125 | 16 |
| 3-109 V1 | 4 | 8 | 0.125 | 16 |
| 3-110 V1 | 4 | 0.125 | 0.125 | 8 |
| 3-111 V1 | 1 | 1 | 0.25 | >128 |
| 3-112 V1 | 3 | 0.5 | 0.25 | >128 |
| 3-125V1 | 2 | >128 | 0.06 | 16 |
| 3-130 V1 | 1 | 0.25 | 0.06 | 8 |
| T3139v | 1 | 1 | 0.03 | 64 |

TABLE 5
Minimal Inhibitory Concentration (MIC) for 62 isolates of Atopobium
vaginae recovered from the vagina between 2009-2012

| Isolate | | Rifaximin | Clindamycin | Metronidazole |
|---|---|---|---|---|
| 600823 | I1 | 0.00375 | 0.03 | 32 |
| 601242 | 21 | 0.00375 | 0.03 | >128 |
| 601255 | 16 | 0.25 | 16 | 64 |
| 601256 | 13 | 0.015 | 0.25 | >128 |
| 601258 | 16 | 0.00375 | 0.03 | >128 |
| 601261 | G | 0.00375 | 0.03 | 128 |
| 601265 | B | 0.00375 | 0.03 | 32 |
| 601278 | 22 | 0.00375 | 0.03 | >128 |
| 601287 | C | 0.00375 | 0.03 | 128 |
| 601291 | 23 | 0.00375 | 0.03 | 128 |
| 601336 | 26 | 0.0075 | 8 | 128 |
| 601342 | D | 0.0075 | 0.25 | >128 |
| 601350 | I | 0.00375 | 0.06 | >128 |
| 601368 | 27 | 0.00375 | 0.06 | 128 |
| 601399 | 11 | 0.00375 | 0.03 | >128 |
| 601401 | R | 0.00375 | 0.03 | >128 |
| 601418 | 17 | 0.0075 | 0.06 | >128 |
| 601422 | 27 | 0.0075 | 0.03 | >128 |
| 601429 | 5 | 0.015 | 0.06 | >128 |
| 601434 | 16 | 0.00375 | 0.03 | 128 |
| 601454 | 10 | 0.125 | 0.03 | 128 |
| 601457 | 13 | 0.00375 | 0.125 | 32 |
| 601486 | 14 | 0.00375 | 0.03 | >128 |
| 601489 | D | 0.0075 | 0.06 | 32 |
| 601505 | 18B | 0.06 | 0.25 | 32 |
| 601508 | C | 0.015 | 0.25 | >128 |
| 601511 | M | 0.00375 | 0.03 | >128 |
| 601518 | A | 0.00375 | 0.03 | >128 |
| 601520 | D | 0.00375 | 0.03 | 8 |
| 601525 | 13 | 0.00375 | 0.06 | >128 |
| 601530 | V | 0.0075 | 0.06 | 32 |
| 602282 | F | 0.00375 | 0.125 | 64 |
| 602286 | D | 0.00375 | 0.125 | 16 |
| 602288 | 1 | 0.00375 | 0.125 | 16 |
| 602290 | F | 0.00375 | 0.125 | 16 |
| 602296 | 8 | 0.0075 | 0.125 | 32 |
| 602302 | 23 | 0.125 | >128 | >128 |
| 602305 | M | 0.015 | 0.125 | 64 |
| 602315 | D | 0.00375 | 0.125 | 64 |
| 602317 | 16 | 0.00375 | 0.125 | 16 |
| 602320 | F | 0.00375 | 0.125 | 4 |
| 602324 | 8B | 0.00375 | 0.125 | 64 |
| 602348 | C | 0.00375 | 0.125 | 16 |
| 602357 | C | 0.00375 | 0.125 | 64 |
| 602364 | C | 0.00375 | 0.125 | 32 |
| 602367 | 11B1 | 0.00375 | 1 | 64 |
| 602368 | E | 0.0075 | 0.125 | 64 |
| 602370 | A | 0.00375 | 0.125 | 8 |
| 602375 | 26 | 0.00375 | 0.03 | 32 |
| 1-001 | 3 | 0.00375 | 0.03 | 128 |
| 1-014 | 12 | 0.00375 | 0.03 | 128 |
| 1-017 | 6 | 0.00375 | 0.03 | 128 |
| 1-029 | 8 | 0.00375 | 0.03 | 128 |
| 1-038 | 4 | 0.00375 | 0.03 | 128 |
| 1-061 | 9 | 0.00375 | 0.03 | >128 |
| 1-064 | 9 | 0.00375 | 0.03 | >128 |
| 1-072 | 3 | 0.00375 | 0.03 | >128 |
| 1-076 | 9 | 0.00375 | 0.03 | >128 |
| 1-081 | 11 | 0.0075 | 0.06 | >128 |
| 1-097 | 23 | 0.0075 | 0.06 | >128 |
| 1-102 | 8 | 0.00375 | 0.03 | >128 |
| 1-106 | 17 | 0.015 | 0.06 | 32 |

TABLE 6
Minimal Inhibitory Concentration (MIC) for 40 isolates of Mobilluncus
species recovered from the vagina between 2009-2012

| Isolate | | species | Rifaximin | Clindamycin | Metronidazole |
|---|---|---|---|---|---|
| 426801 | 8 | M. curtisii | 0.015 | >128 | 0.125 |
| 427518 | G | M. curtisii | 0.015 | >128 | 32 |
| 600719 | J | M. curtisii | 0.015 | >128 | 0.25 |
| 600760 | 7 | M. curtisii | 0.03 | 64 | 0.125 |
| 600813 | H | M. curtisii | 0.015 | >128 | 0.06 |
| 601170 | 21 | M. curtisii | 0.015 | >128 | 0.25 |
| 601195 | 28 | M. curtisii | 0.015 | >128 | 0.25 |
| 601221 | 4 | M. curtisii | 0.015 | >128 | 0.25 |
| 601504 | k | M. curtisii | 0.015 | >128 | 0.25 |
| 601530 | W | M. curtisii | 0.03 | 64 | 0.125 |
| 602367 | 24 | M. curtisii | 0.015 | >128 | 0.06 |
| 1-069 | 5 | M. curtisii | 0.015 | >128 | >128 |
| 1-071 | 2 | M. curtisii | 0.015 | 64 | 0.06 |
| 3-038 | 10 | M. curtisii | 0.015 | >128 | 0.125 |
| 426842 | E | M. mulieris | 0.0075 | 8 | 0.125 |
| 427479 | V | M. mulieris | 0.0075 | >128 | 0.06 |
| 600719 | F | M. mulieris | 0.0075 | 4 | 0.125 |
| 600760 | 3 | M. mulieris | 0.0075 | 2 | 0.06 |
| 600777 | C | M. mulieris | 0.015 | 16 | 0.125 |
| 600796 | S | M. mulieris | 0.0075 | 2 | 0.06 |
| 600842 | 2 | M. mulieris | 0.0075 | 2 | 0.06 |
| 600845 | 1 | M. mulieris | 0.0075 | 8 | 0.125 |
| 600881 | 15 | M. mulieris | 0.015 | 4 | 0.06 |
| 600888 | G | M. mulieris | 0.0075 | 4 | 0.06 |
| 600912 | 2 | M. mulieris | 0.0075 | 8 | 0.125 |
| 601028 | 25 | M. mulieris | 0.0075 | 4 | 0.06 |
| 601105 | 9 | M. mulieris | 0.0075 | 4 | 0.06 |
| 601156 | 19 | M. mulieris | 0.0075 | 8 | 0.125 |
| 601194 | 10 | M. mulieris | 0.0075 | 8 | 0.125 |
| 601279 | N | M. mulieris | 0.0075 | 8 | 0.125 |
| 601336 | 21 | M. mulieris | 0.0075 | 2 | 0.06 |
| 601377 | 11 | M. mulieris | 0.015 | 64 | 0.06 |
| 601450 | 18 | M. mulieris | 0.0075 | 4 | 0.06 |
| 601504 | I | M. mulieris | 0.0075 | 2 | 0.06 |
| 601511 | B | M. mulieris | 0.0075 | 8 | 0.125 |
| 601530 | M | M. mulieris | 0.0075 | 8 | 0.125 |
| 602271 | E | M. mulieris | 0.0075 | 4 | 0.03 |
| 602305 | B | M. mulieris | 0.0075 | >128 | 0.125 |
| 602407 | 20 | M. mulieris | 0.0075 | 8 | 0.125 |
| 3-125 | 6 | Mobiluncus | 0.03 | 4 | 0.03 |
| | | spp | | | |

TABLE 7
Minimal Inhibitory Concentration (MIC) for 33 isolates of Prevotella amnii
recovered from the vagina between 2009-2012

| Isolate | | Rifaximin | Clindamycin | Metronidazole |
|---|---|---|---|---|
| 601203 | 4 | 0.015 | 0.03 | 1 |
| 601256 | 11 | 0.03 | 0.03 | 1 |
| 601258 | 25 | 0.015 | 0.03 | 0.5 |
| 601261 | E | 0.015 | 0.03 | 1 |
| 601266 | 20 | 0.03 | 0.03 | 0.5 |
| 601278 | 10 | 0.015 | 0.03 | 1 |
| 601287 | HH | 0.015 | 0.03 | 0.5 |
| 601291 | 41 | 0.015 | 0.03 | 1 |
| 601293 | 29 | 0.03 | 0.03 | 1 |
| 601305 | A | 0.03 | 0.03 | 2 |
| 601311 | K | 0.03 | 0.03 | 0.5 |
| 601314 | 19 | 0.03 | 0.03 | 2 |
| 601318 | G | 0.03 | 0.03 | 1 |
| 601336 | 33 | 0.015 | 0.03 | 1 |
| 601340 | 37 | 0.015 | 128 | 2 |
| 601341 | H | 0.015 | 0.03 | 1 |
| 601350 | A | 0.015 | 0.03 | 0.5 |
| 601365 | 6 | 0.015 | 0.03 | 0.25 |
| 601368 | 24 | 0.015 | 0.03 | 0.5 |
| 601369 | 13 | 0.03 | 0.03 | 2 |
| 601375 | L | 0.015 | 0.03 | 1 |
| 601377 | 9 | 0.03 | 0.03 | 1 |
| 601379 | 24 | 0.03 | 0.03 | 1 |
| 601388 | G | 0.03 | 0.03 | 1 |
| 601399 | 10 | 0.015 | 0.03 | 1 |
| 601405 | A | 0.015 | 0.03 | 1 |
| 601411 | L | 0.03 | 0.03 | 1 |
| 601413 | 10 | 0.015 | 0.03 | 1 |
| 601418 | 27 | 0.03 | 0.03 | 1 |
| 601426 | A | 0.015 | 0.03 | 1 |
| 601430 | 14 | 0.015 | 0.03 | 1 |
| 601434 | 13 | 0.015 | 0.03 | 0.5 |
| 601458 | 13 | 0.03 | 0.03 | 1 |

TABLE 8
Minimal Inhibitory Concentration (MIC) for 34 isolates of Prevotella
bivia recovered from the vagina between 2009-2012

| Isolate | | Rifaximin | Clindamycin | Metronidazole |
|---|---|---|---|---|
| 601096 | 25 | 0.125 | >128 | 4 |
| 601166 | M | 0.125 | >128 | 16 |
| 601202 | 21 | 0.5 | >128 | 16 |
| 601203 | 5 | 0.125 | 0.03 | 4 |
| 601209 | A1 | 0.125 | >128 | 4 |
| 601212 | 9 | 0.125 | 0.03 | 4 |
| 601213 | 9A | 0.25 | >128 | 16 |
| 601214 | B | 0.25 | >128 | 16 |
| 601241 | 13 | 0.125 | >128 | 4 |
| 601243 | 15 | 0.125 | >128 | 16 |
| 601255 | 10 | 0.25 | >128 | 4 |
| 601261 | C | 0.25 | >128 | 4 |
| 601283 | 20 | 0.25 | >128 | 16 |
| 601285 | 14 | 0.125 | >128 | 4 |
| 601293 | 22 | 0.25 | 0.03 | 8 |
| 601305 | H | 0.125 | >128 | 16 |
| 601340 | 31 | 0.25 | >128 | 4 |
| 601355 | B | 1 | 0.06 | 8 |
| 601367 | M | 0.5 | 0.03 | 8 |
| 601374 | A1 | 0.25 | >128 | 16 |
| 601378 | B | 0.25 | >128 | 16 |
| 601379 | 19 | 0.25 | >128 | 16 |
| 601388 | T | 0.25 | 0.03 | 4 |
| 601389 | 10 | 0.25 | >128 | 4 |
| 601389 | J | 0.25 | 0.03 | 4 |
| 601392 | 14 | 0.125 | >128 | 4 |
| 601409 | V | 0.25 | 0.03 | 4 |
| 601410 | 12 | 0.25 | 0.03 | 4 |
| 601429 | 1 | 0.25 | >128 | 8 |
| 601433 | 19 | 0.25 | >128 | 8 |
| 601449 | E | 0.5 | >128 | 16 |
| 601453 | A | 0.25 | >128 | 8 |
| 601455 | D1 | 0.25 | >128 | 16 |
| 601458 | 16 | 0.25 | >128 | 16 |

TABLE 9
Minimal Inhibitory Concentration (MIC) for 33 isolates of Prevotella
timonensis recovered from the vagina between 2009-2012

| Isolate | | Rifaximin | Clindamycin | Metronidazole |
|---|---|---|---|---|
| 301212 | 10 | 0.00375 | >128 | 2 |
| 601080 | 5 | 0.0075 | 0.03 | 2 |
| 601088 | 15 | 0.015 | 0.03 | 2 |
| 601093 | 24 | 0.015 | 0.03 | 2 |
| 601097 | S | 0.015 | 0.03 | 2 |
| 601098 | B | 0.015 | 0.03 | 2 |
| 601157 | 19 | 0.015 | >128 | 2 |
| 601167 | 13 | 0.06 | 0.06 | 2 |
| 601170 | 13 | 0.015 | 0.03 | 2 |
| 601175 | J | 0.015 | >128 | 2 |
| 601178 | 19 | 0.015 | 0.03 | 2 |
| 601194 | 9B | 0.0075 | 0.03 | 2 |
| 601214 | A | 0.015 | >128 | 4 |
| 601226 | C | 0.015 | >128 | 2 |
| 601234 | B | 0.015 | >128 | 2 |
| 601245 | 16 | 0.015 | 0.03 | 1 |
| 601252 | B | 0.015 | 0.03 | 4 |
| 601255 | 17 | 0.015 | 0.03 | 2 |
| 601266 | 18 | 0.015 | 0.03 | 2 |
| 601278 | 12 | 0.015 | 0.06 | >128 |
| 601281 | 22 | 0.015 | 0.03 | 2 |
| 601287 | H | 0.015 | 0.03 | 2 |
| 601293 | 13 | 0.015 | >128 | 4 |
| 601311 | B | 0.015 | 0.03 | 1 |
| 601367 | E | 0.015 | >128 | 4 |
| 601370 | 13 | 0.03 | >128 | 4 |
| 601388 | S | 0.015 | 0.03 | 4 |
| 601401 | J | 0.015 | >128 | 2 |
| 601411 | S | 0.015 | >128 | 4 |
| 601414 | O | 0.03 | >128 | 4 |
| 601422 | 39 | 0.015 | >128 | 2 |
| 601429 | 11 | 0.015 | 128 | 2 |
| 601459 | 21 | 0.015 | 0.03 | 2 |

TABLE 10
Minimal Inhibitory Concentration (MIC) for 21 isolates of Anaerococcus
tetradius recovered from the vagina between 2009-2012

| Isolate | | Rifaximin | Clindamycin | Metronidazole |
|---|---|---|---|---|
| 601098V | T | 0.06 | 2 | 0.5 |
| 601103V | 21 | 0.06 | 2 | 1 |
| 601175V | I | 0.125 | 1 | 1 |
| 601202V | 11 | 0.06 | 2 | 1 |
| 601212V | 14 | 0.06 | 2 | 1 |
| 601221V | 12 | 0.00375 | 0.03 | 128 |
| 601222V | 15 | 0.06 | 0.25 | 0.5 |
| 601242V | 17 | 0.06 | 4 | 1 |
| 601256V | 9 | 0.06 | 2 | 1 |
| 601287V | V | 0.06 | >128 | 1 |
| 601305V | J | 0.015 | 2 | 1 |
| 601326V | H | 0.06 | 2 | 1 |
| 601340V | 25 | 0.06 | 2 | 1 |
| 601342V | N | 0.06 | 2 | 1 |
| 601368V | 42 | 0.06 | 2 | 1 |
| 601369V | 12 | 0.03 | 2 | 1 |
| 601379V | 25 | 0.06 | >128 | 1 |
| 601401V | T | 0.06 | 8 | 1 |
| 601429V | 15 | 0.015 | 2 | 1 |
| 601450V | 22 | 0.06 | 1 | 1 |
| 602375V | 22 | 0.015 | >128 | 1 |

TABLE 11
Minimal Inhibitory Concentration (MIC) for 20 isolates of Finegoldia
magna recovered from the vagina between 2009-2012

| Isolate | | Rifaximin | Clindamycin | Metronidazole |
|---|---|---|---|---|
| 301303V | D | 4 | 8 | 0.5 |
| 601205V | 39 | 4 | 8 | 4 |
| 601214V | F | 8 | >128 | 2 |
| 601275V | c | 0.125 | 128 | 4 |
| 601276V | 23 | 4 | 0.5 | 2 |
| 601283V | 14 | 4 | 4 | 2 |
| 601293V | 27 | 4 | >128 | 2 |
| 601295V | 25 | 4 | 0.25 | 1 |
| 601298V | 30 | 4 | 128 | 2 |
| 601308V | 24 | 2 | 0.25 | 2 |
| 601315V | 17 | 8 | 4 | 4 |
| 601315V | 17A | 8 | 4 | 4 |
| 601320V | 24 | 16 | 0.5 | 4 |
| 601323V | K | 4 | 0.25 | 0.5 |
| 601330V | c | 16 | 0.25 | 4 |
| 601335V | c1 | 4 | 0.5 | 1 |
| 601340V | 27 | 8 | 0.25 | 4 |
| 601359V | c | 2 | 0.25 | 4 |
| 601363V | 6 | 0.25 | 0.03 | 1 |
| 601367V | c | 16 | 0.5 | 4 |

TABLE 12
Minimal Inhibitory Concentration (MIC) for 23 isolates of Peptoniphilus
harei recovered from the vagina between 2009-2012

| Isolate | | Rifaximin | Clindamycin | Metronidazole |
|---|---|---|---|---|
| 426868V | D | 0.0075 | >128 | 2 |
| 601098V | Q | 0.0075 | 1 | 1 |
| 601203V | 8 | 0.0075 | 0.25 | 2 |
| 601212V | 15 | 0.0075 | 1 | 1 |
| 601233V | R | 0.03 | >128 | 4 |
| 601248V | 23 | 0.0075 | 0.5 | 1 |
| 601259V | 11 | 0.015 | 1 | 2 |
| 601289V | C | 0.0075 | 1 | 2 |
| 601293V | 21 | 0.0075 | 0.25 | 1 |
| 601302V | F1 | 0.015 | 0.5 | 2 |
| 601311V | R | 0.00375 | 0.125 | 1 |
| 601326V | F | 0.015 | 1 | 2 |
| 601342V | F | 0.0075 | 0.125 | 1 |
| 601355V | D | 0.015 | 0.25 | 1 |
| 601365V | 14 | 0.0075 | 0.125 | 1 |
| 601374V | F | 0.015 | 0.5 | 2 |
| 601388V | Q | 0.0075 | 0.5 | 1 |
| 601399V | 16 | 0.0075 | 0.25 | 1 |
| 601409V | L | 0.0075 | 0.25 | 1 |
| 601413V | 16 | 0.0075 | 0.25 | 2 |
| 601426V | D | 0.0075 | 0.125 | 1 |
| 601430V | 22 | 0.015 | 0.125 | 1 |
| 601539V | 25B | 0.0075 | 0.25 | 2 |

TABLE 13
Minimal Inhibitory Concentration (MIC) for 20 isolates of Peptoniphilus
lacrimalis recovered from the vagina between 2009-2012

| Isolate | | Rifaximin | Clindamycin | Metronidazole |
|---|---|---|---|---|
| 601028V | 27A | 0.015 | 128 | 2 |
| 601060V | 32A | 0.015 | 0.125 | 2 |
| 601078V | O | 0.015 | 0.125 | 1 |
| 601084V | c | 0.015 | 0.125 | 4 |
| 601105V | 13 | 0.015 | 0.06 | 4 |
| 601139V | 17 | 0.015 | 128 | 4 |
| 601213V | 17 | 0.015 | >128 | 2 |
| 601255V | 12 | 0.015 | 0.25 | 1 |
| 601261V | O | 0.015 | >128 | 1 |
| 601293V | 15 | 0.03 | 0.25 | 1 |
| 601296V | N | 0.015 | >128 | 1 |
| 601305V | K-1 | 0.0075 | 0.25 | 1 |
| 601318V | E | 0.0075 | 8 | 1 |
| 601330V | H | 0.015 | 0.25 | 1 |
| 601336V | 47 | 0.015 | 0.25 | 1 |
| 601365V | 13 | 0.0075 | 0.125 | 2 |
| 601368V | 39 | 0.0075 | 0.25 | 0.5 |
| 601375V | E | 0.0075 | 0.25 | 1 |
| 601388V | B | 0.00375 | 0.25 | 1 |
| 601401V | I | 0.00375 | 0.25 | 1 |

TABLE 14
Minimal Inhibitory Concentration (MIC) for 25 isolates of Megasphaera-
like bacteria recovered from the vagina between 2009-2012

| Isolate | | Rifaximin | Metronidazole | Clindamycin |
|---|---|---|---|---|
| 426749 | T | 0.0075 | 0.25 | 0.03 |
| 426766 | Q | 0.015 | 0.25 | 0.03 |
| 426788 | C | 0.015 | 0.25 | 0.03 |
| 426788 | 8a | 0.015 | 0.25 | 0.03 |
| 427470 | 10 | 0.015 | 0.125 | 0.03 |
| 427490 | 2u | 0.015 | 0.125 | 0.03 |
| 427507 | H | 0.015 | 0.25 | 0.03 |
| 427509 | A | 0.015 | 0.125 | 0.125 |
| 427522 | 8 | 0.015 | 0.25 | 0.03 |
| 600770 | O | 0.015 | 0.25 | 0.03 |
| 600790 | N | 0.015 | 0.25 | 0.03 |
| 600796 | V | 0.015 | 0.125 | 0.03 |
| 600831 | 9 | 0.015 | 0.25 | 0.03 |
| 600837 | 18 | 0.015 | 0.25 | 0.03 |
| 600861 | P | 0.015 | 0.25 | 0.03 |
| 602236 | E3 | 0.007 | 50.25 | 0.03 |
| 602241 | F | 0.015 | 0.25 | 0.03 |
| 602241 | D | 0.015 | 0.25 | 0.03 |
| 602270 | A | 0.015 | 0.25 | 0.03 |
| 602282 | I | 0.015 | 0.25 | 0.03 |
| 602288 | 16 | 0.015 | 0.125 | 0.03 |
| 602302 | 22 | 0.015 | 0.25 | 0.03 |
| 602305 | I | 0.015 | 0.25 | 0.03 |
| 602324 | 8 | 0.0075 | 0.25 | 0.03 |
| 602672 | 12A | 0.015 | 0.25 | 0.03 |

1. Rifaximin for use in the treatment of a bacterial vaginal infection in a patient, comprising administering a pharmaceutically effective amount of rifaximin to said patient, wherein said bacteria include at least one bacteria strain that is resistant to an antibiotic other than rifaximin.

2. Rifaximin for use in the treatment of a bacterial vaginal infection according to claim 1, wherein said antibiotic is clindamycin or metronidazole.

3. Rifaximin for use in the treatment of a bacterial vaginal infection according to claim 1, wherein the at least one strain of resistant bacteria is selected from the group consisting of Prevotella, Anaerococcus, Finegoldia, Peptoniphilus, Anaerococcus, Peptoniphilus, Megasphera, Mobilincus and Atopobium.

4. Rifaximin for use in the treatment of a bacterial vaginal infection according to claim 1, wherein the at least one strain of resistant bacteria is Prevotella.

5. Rifaximin for use in the treatment of a bacterial vaginal infections according to claim 1, wherein said at least one strain of resistant bacteria is Prevotella bivia.

6. Rifaximin for use in the treatment of a bacterial vaginal infection according to claim 1, wherein the infection is bacterial vaginosis.

7. Rifaximin for use in the treatment of a bacterial vaginal infection according to claim 1, wherein said rifaximin is administered in a daily dosage comprising from 20 to 2000 mg, wherein the dosage form is selected form the group consisting of tablets, coated and uncoated tablets, bioadhesive tablets, controlled release tablet, multi layer tablets, capsules, ointment, cream, gel, foam, and vaginal solutions.

8. Rifaximin for use in the treatment of a bacterial vaginal infection according to claim 6, wherein the daily dose is less than 100 mg.

9. Rifaximin for use in the treatment of a bacterial vaginal infection according to claim 1, wherein said patient is non responsive to clindamycin.

10. Rifaximin for use in the treatment of a bacterial vaginal infection according to claim 1, wherein the patient is non responsive to metronidazole.

11. Rifaximin for use in the treatment of a bacterial vaginal infection in a patient, comprising administering a pharmaceutically effective amount of rifaximin to said patient in combination with one or more additional antibiotics, wherein said bacteria is at least one strain that is resistant or less susceptible to said one or more additional antibiotic.

12. Rifaximin for use in the treatment of a bacterial vaginal infection according to claim 11, wherein said one or more additional antibiotics is administered to said patient is either serially or in conjunction with rifaximin.

13. Rifaximin for use in the treatment of a bacterial vaginal infection according to claim 11, wherein the one or more additional antibiotics is selected from clindamycin and metronidazole.

14. Rifaximin for use in the treatment of a relapse bacterial vaginal infection in a patient, comprising administering a pharmaceutically effective amount of rifaximin to said patient, wherein a previous infection was treated with one or more antibiotics other than rifaximin.

15. Rifaximin for use in the treatment of a relapse bacterial vaginal infection according to claim 14, wherein the infection is bacterial vaginosis.

16. Rifaximin for use in the treatment of a relapse bacterial vaginal infection according to claim 14, wherein said bacteria include at least one bacteria strain that is resistant to said one or more antibiotics used to treat the previous infection.

17. Rifaximin for use in the treatment of a relapse bacterial vaginal infection according to claim 14, wherein said previous infection was treated with clindamycin or metronizazole.

18. Rifaximin for use in the treatment of a relapse bacterial vaginal infection in a patient, in association with clindamycin and metronizazole wherein the therapeutically effective amount selectively reduces an amount of vaginal pathogenic bacteria, including Prevotella strains.

19. Rifaximin for use in the treatment of a bacterial vaginal infection according to claim 18, wherein the infection is bacterial vaginosis.

20. Rifaximin for use in the treatment of a bacterial vaginal infection according to claim 18, wherein the rifaximin is vaginally administered.

21. Rifaximin for use in the treatment of a bacterial vaginal infection according to claim 18, wherein therapeutically effective amount of rifaximin is a daily dose from 20 to 2000 mg.

22. Rifaximin for use in the treatment of a bacterial vaginal infection according to claim 21, wherein therapeutically effective amount of rifaximin is a daily dose is less than 500 mg.

23. Rifaximin for use in the treatment of a bacterial vaginal infection according to claim 21, wherein therapeutically effective amount of rifaximin is a daily dose is less than 100 mg.
